SEQUENCE LISTING (16 sequences)

SEQ ID NO: 1 - DNA, 28 bp, Artificial Sequence
  Primer
ctcggatccggcgcccatcacggcctac

SEQ ID NO: 2 - DNA, 33 bp, Artificial Sequence
  primer
ctctctagatcagcactcttccatttcatcgaa

SEQ ID NO: 3 - DNA, 36 bp, Artificial Sequence
  primer
ctctctagatcagcactcttccatttcatcgaactc

SEQ ID NO: 4 - DNA, 34 bp, Artificial Sequence
  primer
ctcggatccggcgcccatcacggcctactcccaa

SEQ ID NO: 5 - protein, 16 aa, Hepatitis C Virus
PDREVLYREFDEMEEC

SEQ ID NO: 6 - DNA, 28 bp, Artificial Sequence
  primer
ctcggatccggctcccattactgcttac

SEQ ID NO: 7 - DNA, 43 bp, Artificial Sequence
  primer
gacgcgtcgacgcggccgctcagcactcttccatttcactgaa

SEQ ID NO: 8 - DNA, 31 bp, Artificial Sequence
  primer
ctcggatcgggccccgatcacagcatacgcc

SEQ ID NO: 9 - DNA, 41 bp, Artificial Sequence
  primer
caccgctcgagtcagcattcttccatctcatcatattgttg

SEQ ID NO: 10 - protein, 11 aa, Artificial Sequence
  VARIANT 1, 6, 9: Asp at position 1 is linked to anthranilyl
DDIVPXAMXTW

SEQ ID NO: 11 - protein, 11 aa, Artificial Sequence
  VARIANT 6: Xaa at position 6 is aminobutyric acid
DDIVPXAMYTW

SEQ ID NO: 12 - protein, 12 aa, Hepatitis C Virus
DDIVPCSMSYTW

SEQ ID NO: 13 - protein, 11 aa, Artificial Sequence
  VARIANT 1, 2, 6, 9: Xaa is at position 1 is anthranilyl-Asp
XXIVPXAMXTW

SEQ ID NO: 14 - DNA, 30 bp, Artificial Sequence
  primer
cgcatatggcaccttttacgctgcagtgtc

SEQ ID NO: 15 - DNA, 33 bp, Artificial Sequence
  primer
cgcgcgctcgagacactcctccacgatttcttc

SEQ ID NO: 16 - protein, 9 aa, Artificial Sequence
  VARIANT 1, 3, 6, 9: Xaa at position 1 is acetylated-Asp
XEXEEXASX

(19) United States
(12) Patent Application Publication
(10) Pub. No.: US 2005/0159345 A1
(43) Pub. Date: Jul. 21, 2005

(54) Composition for the treatment of infection by Flaviviridae viruses

(75) Inventors: Daniel Lamarre, Laval (CA); Lisette Lagace, Laval (CA)

Correspondence Address: MICHAEL P. MORRIS;BOEHRINGER INGELHEIM CORPORATION, 900 RIDGEBURY RD, P O BOX 368, RIDGEFIELD, CT, 06877-0368, US

(73) Assignee: Boehringer Ingelheim International GmbH, Ingelheim (DE)

(21) Appl. No.: 10/687,204

(22) Filed: Oct. 16, 2003

Related U.S. Application Data
(60) Provisional application No. 60/421,900, filed on Oct. 29, 2002.
(60) Provisional application No. 60/442,769, filed on Jan. 27, 2003.

(51) Int. Cl. A61K 38/12; A61K 31/4709

(52) U.S. Cl. 514/9; 514/312

(57) ABSTRACT

Compositions, use, article of manufacture and method for the treatment of a mammal infected with a virus of the Flaviviridae family are provided comprising administration to the infected mammal of a compound having the Formula I: 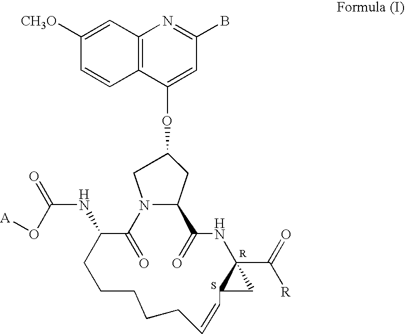 wherein,
A is selected from: C₁ to C₆ alkyl and C₃ to C₆ cycloalkyl; and B is selected from: phenyl or thiazolyl, both of which optionally substituted with a group selected from NH(R¹) and NH(CO)R¹, wherein R¹ is C₁ to C₆ alkyl; R is OH or a sulfonamide derivative; or a pharmaceutically acceptable salt thereof.

### RELATED APPLICATIONS

Benefit of U.S. Provisional Applications, Ser. No. 60/421,900 , filed on Oct. 29, 2002 and Ser. No. 60/442,769, filed on Jan. 27, 2003, is hereby claimed, and said applications are herein incorporated by reference in their entirety.

### FIELD OF THE INVENTION

The present invention relates to compounds, compositions, use and method for the treatment of a Flaviviridae viral infection in a mammal. More particularly, the present invention relates to the use and a method of treatment comprising administration of a compound selected from a macrocyclic peptide family of compounds.

### BACKGROUND OF THE INVENTION

The Flaviviridae family of viruses are enveloped positive-stranded RNA viruses comprising a number of human pathogenic viruses such as viruses of the hepacivirus genus, including Hepatitis C, viruses of the flavivirus genus, including the Dengue Fever viruses, encephalitis viruses, West Nile viruses and Yellow Fever viruses, and viruses of the pestivirus genus, including the bovine viral diarrhea virus and border disease virus, both of which are animal pathogens. The most newly discovered viruses, hepatitis G virus (HGV) and hepatitis GB virus (GBV-A, B, C), are also provisionally considered to be members of the Flaviviridae family belonging to a distinct genus.

Dengue viruses, members of the family of Flaviviridae are transmitted by mosquitos. There are four serotypes that cause widespread human diseases, one of which causes dengue hemorrhagic fever, and about 40% of the population living in tropical and subtropical regions of the world is at risk for infection. Of the 1 million cases of hemorrhagic fever cases per year, about 5% are fatal. There is currently no effective vaccine or antiviral drug to protect against dengue diseases.

Pestiviruses such as bovine diarrhea virus (BVDV), classical swine fever virus (CSFV) and border disease virus (BDV) comprise a group of economically important animal pathogens affecting cattle, pigs and sheep. These positive-sense RNA viruses are classified as a separate genus in the family Flaviviridae.

GB-viruses have been classified as members of the Flaviviridae family, but have not yet been assigned to a particular genus based on the analysis of genomic sequences. These RNA viruses, in addition to infecting humans, can cause acute resolving hepatitis in experimentally infected tamarins.

Viruses within the Flaviviridae family possess a number of similarities despite a relatively low overall sequence homology among its members. The genome of these viruses is a small single-stranded RNA (≈10 kilobases in length) having a single open reading frame (ORF). The ORF encodes a polyprotein of about 3000 amino acids that contains structural proteins at its 5′ end and non-structural (NS) proteins at its 3′ end. The polyprotein is proteolytically processed by both viral and host-encoded proteases into mature polypeptides, which for HCV, are as follows: NH₂—{C-E1-E2-P7-NS2-NS3-NS4A-NS4B-NS5A-NS5B}—COOH. The core protein or nucleocapsid (C) and the two envelope glycoproteins (E1 and E2) represent the constitutive structural proteins of the virions. These structural proteins are followed by the nonstructural (NS) proteins, which at least some are thought to be essential for viral RNA replication.

Enzymatic activities have been ascribed to several of these NS proteins. In particular, the NS3 protein contains sequences with similarity to the serine protease and the nucleoside triphosphate-binding helicase of pestiviruses and flaviviruses. Analysis of sequence alignments had predicted the existence of a trypsin-like serine protease domain within the N-terminal region of flavi-, pesti-, hepaci- and GB-viruses. Sequence similarities between NS3 proteolytic domains of Flaviviridae viruses are well-established (Ryan MD et al. 1998). The HCV NS3 protease domain shares a sequence similarity of about 77-90% among HCV genotypes and a sequence similarity of about 25-50% with other members of the Flaviviridae family. With respect to the three dimensional structure, the available atomic co-ordinates of the various crystallized HCV and Dengue NS3 proteases show an overall architecture that is characteristic of the trypsin-like fold (Kim et al. 1996, Love et al. 1996; Murthy et al. 1999). Many studies have now firmly established that the N-terminal portion of the NS3 region encodes a serine protease that has a very specific and pivotal role in viral polyprotein processing within Flaviviridae. In hepacivirus, pestivirus and GB viruses, polyprotein processing shows a requirement for the downstream NS4A protein. The NS4A protein acts as a cofactor that enhances the NS3 protease cleavage efficiency (Lin et al., 1995; Kim et al., 1996; Steinkuler et al., 1996). In flavivirus, this requirement for enhancing the NS3 protease activity is provided by the upstream NS2B protein. The genomic organization and structure of GBV-B and HCV are similar despite the fact that the sequence homology between the polyprotein sequences of GBV-B and HCV is about 25 to 30%.

Given apparent similarities of viruses within the Flaviviridae family of viruses, it would be desirable to develop therapeutic agents effective against Flaviviridae viruses, and more particularly, effective against the pathogenic members of the Flaviviridae family.

### SUMMARY OF THE INVENTION

Accordingly, in a first aspect of the present invention, there is provided an anti-Flaviviridae virus composition comprising a pharmaceutically acceptable carrier in combination with a compound of Formula (I): 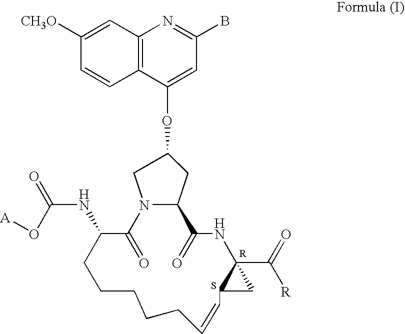
wherein,
A is selected from: C₁ to C₆ alkyl and C₃ to C₆ cycloalkyl; and B is selected from: phenyl or thiazolyl, both of which optionally substituted with a group selected from NH(R¹) and NH(CO)R¹, wherein R¹ is C₁ to C₆ alkyl; R is OH or a sulfonamide derivative, or
a pharmaceutically acceptable salt thereof.

In a second aspect, the present invention provides a method for treating a mammal infected with a virus of the Flaviviridae family comprising administering to the infected mammal a pharmaceutical composition comprising a pharmaceutically acceptable carrier in combination with a therapeutically effective amount of a compound of Formula (I) as defined above.

In a third aspect, the present invention provides a method of treating a mammal infected with a virus of the Flaviviridae family wherein a pharmaceutical composition comprising a pharmaceutically acceptable carrier in combination with a therapeutically effective amount of a compound of Formula (I) as defined above is co-administered with at least one additional agent selected from: an antiviral agent, an immunomodulatory agent, an HCV inhibitor, an HIV inhibitor, an HAV inhibitor and an HBV inhibitor; to the infected mammal.

In a fourth aspect, the present invention provides a pharmaceutical composition for treating or preventing an infection of a mammal caused by a virus of the Flaviviridae family comprising a pharmaceutically acceptable carrier in combination with a therapeutically effective amount of a compound of Formula (I) and at least one additional agent selected from: an antiviral agent, an immunomodulatory agent, an HCV inhibitor, an HIV inhibitor, an HAV inhibitor and an HBV inhibitor.

In a fifth aspect of the present invention, there is provided the use of a compound of Formula (I) as defined above, for the manufacture of a medicament for the treatment of Flaviviridae viral infection.

In a sixth aspect of the present invention, there is provided an article of manufacture comprising packaging material contained within which is a composition effective to inhibit a virus of the Flaviviridae family and the packaging material comprises a label which indicates that the composition can be used to treat infection by a virus of the Flaviviridae family and, wherein said composition comprises a compound of Formula (I) as defined above.

While not wishing to be bound to any particular mode of action, the macrocyclic peptide family of compounds generally represented by Formula (I) as set out above are believed to interact at a conserved substrate binding site in the NS3 protease domain. Crystal structure studies confirm that the present macrocyclic compounds interact at the substrate-binding site within the HCV NS3 domain, a region that is functionally conserved among Flaviviridae viruses.

Other objects, advantages and features of the present invention will become more apparent upon reading the following non-restrictive description of the preferred embodiments with reference to the accompanying drawings and tables in which:

### BRIEF DESCRIPTION OF TABLES

Tables 4-8 provide a sequence similarity comparison between members of the Flaviviridae family.

### BRIEF DESCRIPTION OF THE DRAWINGS

FIG. 1 provides a comparison of polyproteins of viruses belonging to the Flaviviridae family (taken from: Ryan et al., 1998, J. Gen. Virology 79, 947-959);

FIG. 2 is a sequence comparison of the NS3 protease domain between HCV genotypes and subtypes;

FIGS. 3A-B are the IC₅₀ curves of a macrocyclic peptide compound according to Formula (I) against HCV genotype 1a and 1b NS3-NS4A proteases, respectively;

FIGS. 4A-B are the Dixon and Cornish-Bowden plots of the macrocyclic peptide of FIG. 3 against the HCV genotype 1a NS3-NS4A proteases;

FIGS. 5A-B are the Dixon and Cornish-Bowden plots of the macrocyclic peptide of FIG. 3 against the HCV genotype 1b NS3-NS4A proteases;

FIG. 6 illustrates the inhibition of GBV-B replication by macrocyclic peptides according to Formula (I) in tamarin hepatocytes in culture;

FIG. 7 graphically illustrates dose-dependent inhibition of GBV-B replication by the macrocyclic peptide III of FIG. 6; and

FIG. 8 illustrates the 3-dimensional crystal structure of the HCV NS3-NS4A peptide structure complexed with a macrocyclic peptide according to Formula (I).

### DETAILED DESCRIPTION OF THE INVENTION

Definitions

Unless defined otherwise, the scientific and technological terms and nomenclature used herein have the same meaning as commonly understood by a person of ordinary skill in the art to which this invention pertains. Generally, the procedures for cell culture, infection, molecular biology methods and the like are common methods used in the art. Such standard techniques can be found in reference manuals such as for example Sambrook et al. (1989) and Ausubel et al. (1994).

The term “Flaviviridae” as it is used herein to designate a viral family is meant to encompass viruses of the hepacivirus genus, such as Hepatitis C, viruses of the flavivirus genus, such as the Dengue Fever viruses, Encephalitis viruses, West Nile viruses and Yellow Fever viruses, and viruses of the pestivirus genus, such as the bovine viral diarrhea virus and border disease virus. Hepatitis G virus (HGV) and Hepatitis GB virus are also included in this viral family although the genus of these viruses has not yet been determined. Moreover, all subtypes and genotypes of the above-mentioned viruses are also encompassed within the Flaviviridae family, including for example, HCV 1a, HCV1b, HCV 2a-c, HCV 3a-b, HCV 4a, HCV 5 and HCV 6a,h,d & k, as well as GBV-A, B & C.

The term “mammal” as it is used herein is meant to encompass humans, as well as non-human mammals which are susceptible to infection by a Flaviviridae virus including domestic animals, such as cows, pigs, horses, dogs and cats, and sheep.

With respect to the compounds of Formula (I) administered in the treatment of Flaviviridae infection, the term “C₁₋₆ alkyl” or “C₁-C₆” as used herein, either alone or in combination with another substituent, means acyclic, straight or branched chain alkyl substituents containing from one to six carbon atoms and includes, for example, methyl, ethyl, propyl, isopropyl, butyl, tert-butyl, hexyl, 1-methylethyl, 1-methylpropyl, 2-methylpropyl, 1,1-dimethylethyl.

The term “C₃₋₆ cycloalkyl” as used herein, either alone or in combination with another substituent, means a cycloalkyl substituent containing from three to six carbon atoms and includes cyclopropyl, cyclobutyl, cyclopentyl, and cyclohexyl.

The term “pharmaceutically acceptable salt” means a salt of a compound of formula (I) which is, within the scope of sound medical judgment, suitable for use in contact with the tissues of humans and lower animals without undue toxicity, irritation, allergic response, and the like, commensurate with a reasonable benefit/risk ratio, generally water or oil-soluble or dispersible, and effective for their intended use. The term includes pharmaceutically-acceptable acid addition salts and pharmaceutically-acceptable base addition salts. Lists of suitable salts are found in, e.g., S. M. Birge et al. J. Pharm. Sci., 1977, 66, pp. 1-19, which is hereby incorporated by reference in its entirety.

The term “pharmaceutically-acceptable acid addition salt” means those salts which retain the biological effectiveness and properties of the free bases and which are not biologically or otherwise undesirable, formed with inorganic acids such as hydrochloric acid, hydrobromic acid, hydroiodic acid, sulfuric acid, sulfamic acid, nitric acid, phosphoric acid, and the like, and organic acids such as acetic acid, trichloroacetic acid, trifluoroacetic acid, adipic acid, alginic acid, ascorbic acid, aspartic acid, benzenesulfonic acid, benzoic acid, 2-acetoxybenzoic acid, butyric acid, camphoric acid, camphorsulfonic acid, cinnamic acid, citric acid, digluconic acid, ethanesulfonic acid, glutamic acid, glycolic acid, glycerophosphoric acid, hemisulfic acid, heptanoic acid, hexanoic acid, formic acid, fumaric acid, 2-hydroxyethanesulfonic acid (isethionic acid), lactic acid, maleic acid, hydroxymaleic acid, malic acid, malonic acid, mandelic acid, mesitylenesulfonic acid, methanesulfonic acid, naphthalenesulfonic acid, nicotinic acid, 2-naphthalenesulfonic acid, oxalic acid, pamoic acid, pectinic acid, phenylacetic acid, 3-phenylpropionic acid, picric acid, pivalic acid, propionic acid, pyruvic acid, pyruvic acid, salicylic acid, stearic acid, succinic acid, sulfanilic acid, tartaric acid, p-toluenesulfonic acid, undecanoic acid, and the like.

The term “pharmaceutically-acceptable base addition salt” means those salts which retain the biological effectiveness and properties of the free acids and which are not biologically or otherwise undesirable, formed with inorganic bases such as ammonia or hydroxide, carbonate, or bicarbonate of ammonium or a metal cation such as sodium, potassium, lithium, calcium, magnesium, iron, zinc, copper, manganese, aluminum, and the like. Particularly preferred are the ammonium, potassium, sodium, calcium, and magnesium salts. Salts derived from pharmaceutically-acceptable organic nontoxic bases include salts of primary, secondary, and tertiary amines, quaternary amine compounds, substituted amines including naturally occurring substituted amines, cyclic amines and basic ion-exchange resins, such as methylamine, dimethylamine, trimethylamine, ethylamine, diethylamine, triethylamine, isopropylamine, tripropylamine, tributylamine, ethanolamine, diethanolamine, 2-dimethylaminoethanol, 2-diethylaminoethanol, dicyclohexylamine, lysine, arginine, histidine, caffeine, hydrabamine, choline, betaine, ethylenediamine, glucosamine, methylglucamine, theobromine, purines, piperazine, piperidine, N-ethylpiperidine, tetramethylammonium compounds, tetraethylammonium compounds, pyridine, N,N-dimethylaniline, N-methylpiperidine, N-methylmorpholine, dicyclohexylamine, dibenzylamine, N,N-dibenzylphenethylamine, 1-ephenamine, N,N′-dibenzylethylenediamine, polyamine resins, and the like. Particularly preferred organic nontoxic bases are isopropylamine, diethylamine, ethanolamine, trimethylamine, dicyclohexylamine, choline, and caffeine.

The term “sulfonamide derivative” as used herein means a sulfonamide group of the formula —NHSO₂—R² wherein R² is —(C₁₋₈)alkyl, —(C₃₋₇)cycloalkyl or {—(C₁₋₆)alkyl-(C₃₋₆)cycloalkyl}, which are all optionally substituted from 1 to 3 times with halo, cyano, nitro, O—(C₁₋₆)alkyl, amido, amino or phenyl, or R² is C₆ or C₁₀ aryl which is optionally substituted from 1 to 3 times with halo, cyano, nitro, (C₁₋₆)alkyl, O—(C₁₋₆)alkyl, amido, amino or phenyl.

The term “antiviral agent” as used herein means an agent (compound or biological) that is effective to inhibit the formation and/or replication of a virus in a mammal. This includes agents that interfere with either host or viral mechanisms necessary for the formation and/or replication of a virus in a mammal. Antiviral agents include, for example, ribavirin, amantadine, VX-497 (merimepodib, Vertex Pharmaceuticals), VX-498 (Vertex Pharmaceuticals), Levovirin, Viramidine, Ceplene (maxamine), XTL-001 and XTL-002 (XTL Biopharmaceuticals).

The term “immunomodulatory agent” as used herein means those agents (compounds or biologicals) that are effective to enhance or potentiate the immune system response in a mammal. Immunomodulatory agents include, for example, class I interferons (such as α-, β- and omega interferons, tau-interferons, consensus interferons and as asialo-interferons), class 11 interferons (such as γ-interferons) and pegylated interferons.

The term “inhibitor of HCV NS3 protease” as used herein means an agent (compound or biological) that is effective to inhibit the function of HCV NS3 protease in a mammal. Inhibitors of HCV NS3 protease include, for example, those compounds described in WO 99/07733, WO 99/07734, WO 00/09558, WO 00/09543 or WO 00/59929, WO 03/064416; WO 03/064455; WO 03/064456 and the Vertex pre-development candidate identified as VX-950.

The term “HCV inhibitor” as used herein means an agent (compound or biological) that is effective to inhibit the formation and/or replication of HCV in a mammal. This includes agents that interfere with either host or HCV viral mechanisms necessary for the formation and/or replication of HCV in a mammal. Inhibitors of HCV include, for example, agents that inhibit a target selected from: NS3 protease, NS3 helicase, HCV polymerase, NS2/3 protease or IRES. Specific examples of inhibitors of HCV include ISIS-14803 (ISIS Pharmaceuticals).

The term “inhibitor of HCV polymerase” as used herein means an agent (compound or biological) that is effective to inhibit the function of an HCV polymerase in a mammal. This includes, for example, inhibitors of HCV NS5B polymerase. Inhibitors of HCV polymerase include non-nucleosides, for example, those compounds described in:
U.S. application Ser. No. 10/198,680 filed 18 Jul. 2002, herein incorporated by reference in its entirety, which corresponds to WO 03/010140 (Boehringer Ingelheim),
U.S. application Ser. No. 10/198,384 filed 18 Jul. 2002, herein incorporated by reference in its entirety, which corresponds to WO 03/010141 (Boehringer Ingelheim),
U.S. application Ser. No. 10/198,259 filed 18 Jul. 2002, herein incorporated by reference in its entirety, which corresponds to WO 03/007945 (Boehringer Ingelheim),
WO 02/100846 A1 and WO 02/100851 A2 (both Shire),
WO 01/85172 A1 and WO 02/098424 A1 (both GSK),
WO 00/06529 and WO 02/06246 A1 (both Merck),
WO 01/47883 and WO 03/000254 (both Japan Tobacco) and
EP 1 256 628 A2 (Agouron).
   Furthermore other inhibitors of HCV polymerase also include nucleoside analogs, for example, those compounds described in:
WO 01/90121 A2 (Idenix),
WO 02/069903 A2 (Biocryst Pharmaceuticals Inc.), and
WO 02/057287 A2 and WO 02/057425 A2 (both Merck/Isis).

Specific examples of inhibitors of an HCV polymerase, include JTK-002/003, JTK-109 (Japan Tobacco), and NM283 (Idenix).

The term “HIV inhibitor” as used herein means an agents (compound or biological) that is effective to inhibit the formation and/or replication of HIV in a mammal. This includes agents that interfere with either host or viral mechanisms necessary for the formation and/or replication of HIV in a mammal. HIV inhibitors include, for example, nucleosidic inhibitors, non-nucleosidic inhibitors, protease inhibitors, fusion inhibitors and integrase inhibitors.

The term “HAV inhibitor” as used herein means an agent (compound or biological) that is effective to inhibit the formation and/or replication of HAV in a mammal. This includes agents that interfere with either host or viral mechanisms necessary for the formation and/or replication of HAV in a mammal. HAV inhibitors include Hepatitis A vaccines, for example, Havrix® (GlaxoSmithKline), VAQTA® (Merck) and Avaxim® (Aventis Pasteur).

The term “HBV inhibitor” as used herein means an agent (compound or biological) that is effective to inhibit the formation and/or replication of HBV in a mammal. This includes agents that interfere with either host or viral mechanisms necessary for the formation and/or replication of HBV in a mammal. HBV inhibitors include, for example, agents that inhibit HBV viral DNA polymerase or HBV vaccines. Specific examples of HBV inhibitors include Lamivudine (Epivir-HBV®), Adefovir Dipivoxil, Entecavir, FTC (Coviracil®), DAPD (DXG), L-FMAU (Clevudine®), AM365 (Amrad), Ldt (Telbivudine), monoval-LdC (Valtorcitabine), ACH-126,443 (L-Fd4C) (Achillion), MCC478 (Eli Lilly), Racivir (RCV), Fluoro-L and D nucleosides, Robustaflavone, ICN 2001-3 (ICN), Bam 205 (Novelos), XTL-001 (XTL), Imino-Sugars (Nonyl-DNJ) (Synergy), HepBzyme; and immunomodulator products such as: interferon alpha 2b, HE2000 (Hollis-Eden), Theradigm (Epimmune), EHT899 (Enzo Biochem), Thymosin alpha-1 (Zadaxin®), HBV DNA vaccine (PowderJect), HBV DNA vaccine (Jefferon Center), HBV antigen (OraGen), BayHep B® (Bayer), Nabi-HB® (Nabi) and Anti-hepatitis B (Cangene); and HBV vaccine products such as the following: Engerix B, Recombivax HB, GenHevac B, Hepacare, Bio-Hep B, TwinRix, Comvax, Hexavac.

The term “class I interferon” as used herein means an interferon selected from a group of interferons that all bind to receptor type I. This includes both naturally and synthetically produced class I interferons. Examples of class I interferons include α-, β-, δ-, omega interferons, tau-interferons, consensus interferons, asialo-interferons.

The term “class II interferon” as used herein means an interferon selected from a group of interferons that all bind to receptor type II. Examples of class II interferons include γ-interferons.

Moreover, the term “therapeutically effective amount” as it is used herein with respect to compounds of Formula I means an amount of the compound which is effective to treat a Flaviviridae viral infection, i.e. to inhibit or at least reduce viral replication, while not being an amount that is toxic to a mammal being treated or an amount that may otherwise cause significant adverse effects in a mammal.

The term “carrier” is used to refer to compounds or mixtures of compounds for combination with the present therapeutic compounds which facilitate the administration thereof and/or enhance the function of the therapeutic compounds to inhibit a virus of the Flaviviridae family including, for example, diluents, excipients, adjuvants and vehicles. Stabilizers, colorants, flavorants, anti-microbial agents, and the like may also be combined with the therapeutic compound. Moreover, in some cases, the pH of the formulation may be adjusted with pharmaceutically acceptable acids, bases or buffers to enhance the stability of the formulated compound or its delivery form. Other suitable additives include those with which one of skill in the art would be familiar that function to improve the characteristics of the present combination while not adversely affecting its function. Reference may be made to “Remington's Pharmaceutical Sciences”, 17th Ed., Mack Publishing Company, Easton, Pa., 1985, for carriers that would be suitable for admixture with the present therapeutic compounds. The term “pharmaceutically acceptable” as it is used herein with respect to a carrier compound is meant to indicate that the carrier is suitable for administration to a mammal, i.e. is non-toxic and does not cause any adverse effect when used in amounts appropriate to function as a carrier.

### Preferred Embodiments

Anti-Flaviviridae Virus Composition

In a first embodiment of the present invention, there is provided a composition effective to treat a mammal infected with a virus of the Flaviviridae family. The composition comprises a pharmaceutically acceptable carrier in combination with a compound of Formula (I): 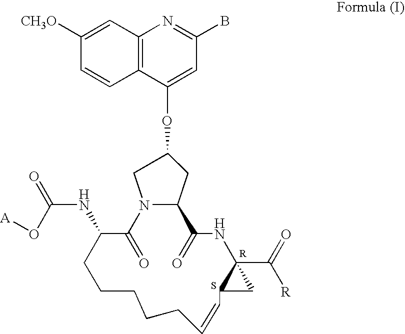
wherein,
A is selected from: C₁ to C₆ alkyl and C₃ to C₆ cycloalkyl; and B is selected from: phenyl or thiazolyl, both of which optionally substituted with a group selected from NH(R¹) and NH(CO)R¹, wherein R¹ is C₁ to C₆ alkyl; R is OH or a sulfonamide derivative, or
a pharmaceutically acceptable salt thereof.

In one embodiment, A of Formula (I) is a branched C₄ to C₆ alkyl or C₄ to C₆ cycloalkyl group. In a preferred embodiment, A is cyclopentyl or tert-butyl.

In another embodiment, B of Formula (I) is phenyl or a thiazole substituted at position 2 with NH(R¹) or NH(CO) R¹ in which R¹ is a C₁ to C₄ alkyl. In a preferred embodiment, B is a thiazole substituted at position 2 with NH(R¹) or NH(CO) R¹ in which R¹ is a C₁ to C₄ alkyl. More preferably, B is 4-thiazole substituted at position 2 with NH(CO)CH₃ or with NHCH(CH₃)₂.

In a further preferred embodiment, R of formula (I) is OH or a sulfonamide group of formula —NHSO₂—R² wherein R² is —(C₁₋₆)alkyl, —(C₃₋₆)cycloalkyl, both optionally substituted 1 or 2 times with halo or phenyl, or R² is C₆ aryl optionally substituted from 1 or 2 times with halo or (C₁₋₆)alkyl. More preferably, R is OH or a sulfonamide group wherein R² is methyl, cyclopropyl or phenyl.

In a more preferred embodiment, the present invention is conducted with a compound of Formula (I) in which A is tert-butyl and B is phenyl as set out below in the formula of compound (II):

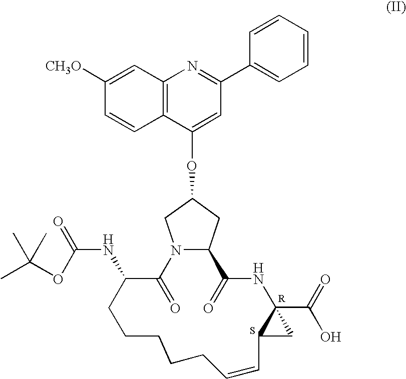

In another more preferred embodiment, the present invention is conducted with a compound of Formula (I) in which A is tert-butyl and B is 4-thiazole substituted at its 2 position with NH(CO)CH₃ as set out below in the formula for compound (III):

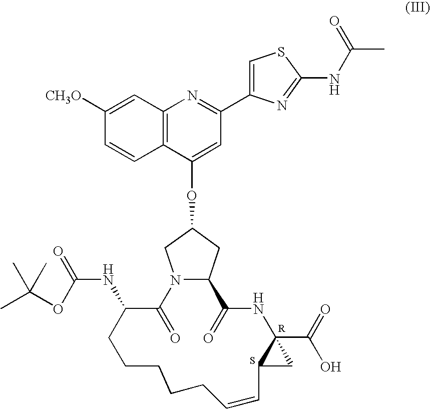

In a most preferred embodiment, the present invention is conducted with a compound of Formula (I) in which A is cyclopentyl and B is 4-thiazole substituted at its 2 position with NHCH(CH₃)₂ as set out below in the formula for compound (IV):

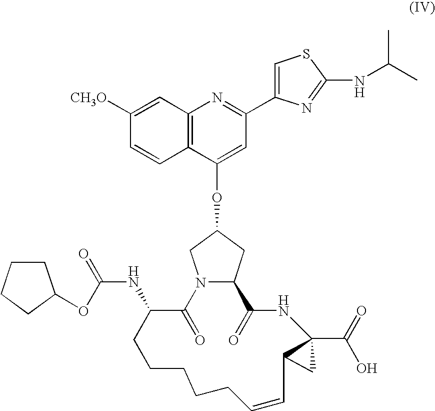

In a most preferred embodiment, the present invention is conducted with a compound of Formula (I) in which A is cyclopentyl, B is 4-thiazole substituted at its 2 position with NHCH(CH₃)₂, and R is a sulfonamide group wherein R² is phenyl as set out below in the formula for compound (VI):

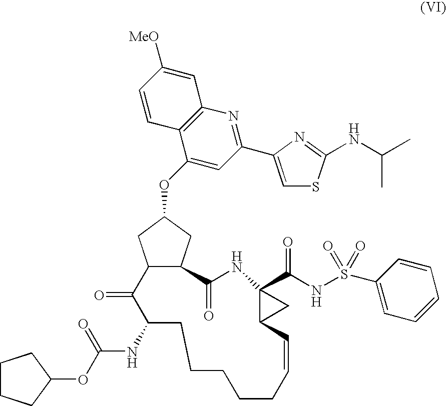

In a most preferred embodiment, the present invention is conducted with a compound of Formula (I) in which A is cyclopentyl, B is 4-thiazole substituted at its 2 position with NHCH(CH₃)₂, and R is a sulfonamide group wherein R² is methyl as set out below in the formula for compound (VII):

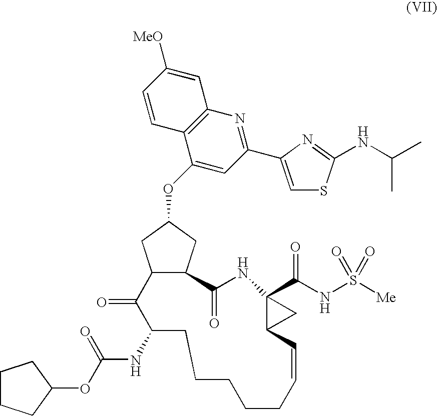

In a most preferred embodiment, the present invention is conducted with a compound of Formula (I) in which A is cyclopentyl, B is 4-thiazole substituted at its 2 position with NHCH(CH₃)₂, and R is a sulfonamide group wherein R² is cyclopropyl as set out below in the formula for compound (VIII): 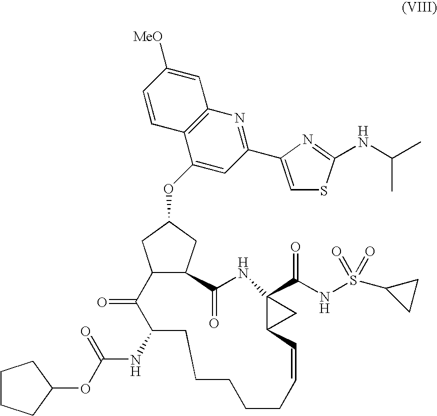
Method of Treatment

In a second aspect of the present invention, a method for treating a mammal infected with a virus of the Flaviviridae family is provided comprising administering to an infected mammal a pharmaceutical composition comprising a pharmaceutically acceptable carrier in combination with a therapeutically effective amount of a compound having Formula (I), (II), (III), (IV), (VI), (VII) or (VIII) as defined above.

In accordance with the method of the present invention, a therapeutically effective amount of a compound of Formula (I), (II), (III), (IV), (VI), (VII) or (VIII) is administered to a mammal infected with a Flaviviridae virus. To be therapeutically effective, a dosage of between about 0.01 and about 100 mg/kg body weight per day, preferably between about 0.1 and about 50 mg/kg body weight per day of the compound is administered to the infected mammal. Typically, the method will involve administration of the compound from about 1 to about 5 times per day or alternatively, as a continuous infusion. Such administration can be used as a chronic or acute therapy.

As one of skill in the art will appreciate, lower or higher doses than those recited above may be required. Specific dosage and treatment regimens will depend upon a variety of factors, including the activity of the specific compound employed, the age, body weight, general health status, sex and diet of the infected mammal, the time of administration, the rate of excretion, the severity and course of the infection, the patient's disposition to the infection and the judgment of the treating physician. Generally, treatment is initiated with small dosages substantially less than the optimum dose of the peptide. Thereafter, the dosage is increased by small increments until the optimum effect under the circumstances is reached. In general, the compound is most desirably administered at a concentration level that will generally afford a therapeutic effect without causing any harmful or deleterious side effects.

Administration of the therapeutic compound in the treatment of Flaviviridae viral infection may be by any one of several routes including administration orally, parenterally or via an implanted reservoir. The term “parenteral” as used herein includes subcutaneous, intracutaneous, intravenous, intramuscular, intra-articular, intrasynovial, intrasternal, intrathecal, and intralesional injection or infusion techniques. Oral administration or administration by injection are preferred. Orally acceptable dosage forms include, but not limited to, capsules, tablets, and aqueous suspensions and solutions.

As set out above, the compound of Formula (I), (II), (III), (IV), (VI), (VII) or (VIII) is administered in combination with one or more pharmaceutically acceptable carriers. The nature of the carrier(s) will, of course, vary with the dosage form. Accordingly, in the case of tablets for oral use, carriers which are commonly used include lactose and corn starch. Lubricating agents, such as magnesium stearate, are also typically added. For oral administration in a capsule form, useful diluents include lactose and dried corn starch. When aqueous suspensions are administered orally, the active ingredient is combined with emulsifying and suspending agents. If desired, certain sweetening and/or flavoring and/or coloring agents may be added. Injectable preparations including injectable aqueous or oleaginous suspensions, are formulated according to techniques known in the art using suitable dispersing or wetting agents such as Tween 80 and suspending agents.

The amount of the present therapeutic compound that is combined with the carrier materials to produce a single dosage form will vary depending upon the host treated and the particular mode of administration. A typical preparation will contain from about 5% to about 95% therapeutic compound (w/w). Preferably, such preparations contain from about 20% to about 80% therapeutic compound.

In another aspect, a combination therapy is contemplated wherein the compound of Formula (I), (II), (III), (IV), (VI), (VII) or (VIII), or a pharmaceutically acceptable salt thereof, is co-administered with at least one additional agent selected from: an antiviral agent, an immunomodulatory agent, an HCV inhibitor, an HIV inhibitor, an HAV inhibitor, an HBV inhibitor, and a therapeutic effective to treat the symptoms of the viral infection. Examples of such agents are well known to those of skill in the art. These additional agents may be combined with the compounds of this invention to create a single pharmaceutical dosage form. Alternatively these additional agents may be separately administered to the infected patient as part of a multiple dosage form, for example, using a kit. Such additional agents may be administered to the patient prior to, concurrently with, or following the administration of the therapeutic compound of Formula (I), (II), (III), (IV), (VI), (VII) or (VIII), or a pharmaceutically acceptable salt thereof.

When a combination therapy is utilized, both the present compound and the additional therapeutic and/or prophylactic agents should be present at dosage levels of between about 10 to 100%, and more preferably between about 10 and 80% of the dosage normally administered in a monotherapy regimen.

Article of Manufacture

In a further aspect of the present invention, there is provided an article of manufacture comprising packaging material contained within which is a composition effective to treat a mammal infected with a virus of the Flaviviridae family and the packaging material comprises a label which indicates that the composition can be used to treat infection by a virus of the Flaviviridae family, wherein said composition comprises a compound of Formula (I), (II), (III), (IV), (VI), (VII) or (VIII) as defined above.

Flaviviridae Viruses

Particularly, the different embodiments of the present invention may be directed to distinct viruses, particularly viruses which are pathogenic in mammal. Preferably, the above-mentioned compounds of compositions can be used for the treatment of hepacivirus genus, such as Hepatitis C. Preferred examples of HCV viruses are selected from genotypes: 1, 2, 3, 4, 5, and 6. More preferred examples of HCV viruses are selected from genotypes: 2, 3, 4, 5, and 6. Preferably, HCV viruses are selected from subtypes: HCV 1a, HCV1b, HCV 2a-c, HCV 3a-b, HCV 4a, HCV 5 and HCV 6a,h,d & k. More preferably, HCV viruses are selected from subtypes: HCV 1a, HCV 2a-c, HCV 3a-b, HCV 4a, HCV 5 and HCV 6a,h,d & k.

Alternatively, the compounds of the invention may be directed against viruses of the flavivirus genus, such as the Dengue Fever viruses, Japanese Encephalitis viruses, West Nile viruses and Yellow Fever viruses. Preferably, the invention is directed at the treatment of viral disease in a human caused by Dengue virus. Preferably, the invention is directed at the treatment of viral disease in a human caused by Japanese encephalitis virus. Alternatively, the invention is directed at the treatment of viral disease in a human caused by Yellow Fever virus. Alternatively, the invention is directed at the treatment of viral disease in a human caused by West Nile virus.

In addition, the invention may be directed to the treatment of viral disease caused by viruses of the pestivirus genus, such as the bovine viral diarrhea virus (BVDV), classical swine fever virus (CSFV) and border disease virus (BDV). Preferably, the invention is directed at the treatment of viral diseases in cattle caused by BVDV. Alternatively, the invention is directed at the treatment of viral diseases in pigs caused by CSFV. Alternatively, the invention is directed at the treatment of viral diseases in sheep caused by BDV.

Furthermore, GB viruses can also be treated with composition of the present invention. Included in this viral family are Hepatitis G virus (HGV) and Hepatitis GB virus. Preferred examples are selected from: GBV-A, B & C. Preferably, the invention is directed at the treatment of viral disease in a human caused by GBV-C. Alternatively, the invention is directed at the treatment of viral disease in a human caused by GBV-A. Alternatively, the invention is directed at the treatment of viral disease in a human caused by HGV.

Embodiments of the present invention are exemplified by the following specific examples which are not to be construed as limiting.

### EXAMPLES

Abbreviations used in the examples include:
Abu: aminobutyric acid; DABCYL: 4-((4-(dimethylamino)phenyl) azo)benzoic acid; DMEM: Dulbecco's Modified Eagle Medium; DMSO: dimethyl sulfoxide; EDANS: 5-((2-aminoethyl)amino)naphthalene-1-sulfonic acid; HPLC: high performance liquid chromatography; IPTG: isopropyl-b-D-thiogalactoside; LB: Luria-Bertoni (as in LB broth); Nva: norvaline; PenStrep: penicillin/streptomycin; PCR: polymerase chain reaction; r.m.s.: root mean square; RT-PCR: real-time polymerase chain reaction; and TCEP: tris(2-carboxyethyl)phosphine hydrochloride.
   Synthesis of Compounds of Formula (I)

Compounds of Formula (I) were prepared using the protocol outlined in detail in WO 00/059929, published Oct. 12, 2000, the contents of which are incorporated herein by reference. In particular, reference is made to page 89, Example 34C for the preparation of compound (IV).

### Example 1

### Inhibition of HCV NS3-NS4A Proteases of Different Genotypes by Compounds II, III and IV

HCV NS3/4A 1a and 1b

For production of the HCV genotype 1b NS3-NS4A heterodimer protein, a full-length HCV cDNA was cloned by RT-PCR using RNA extracted from the serum of an HCV genotype 1b infected individual (provided by Dr. Bernard Willems, Hôpital St-Luc, Montréal, Canada). The DNA region encoding the NS3-NS4A heterodimer protein was PCR-amplified (forward primer: ′CTCGGATCCGGCGCCCATCACGGCCTAC3′ (SEQ ID No.1); reverse primer: 5′CTCTCTAGATCAGCACTCTTCCATTTCAT CGM3′) (SEQ ID No.2)) from the full-length HCV cDNA and subcloned into the pFastBac™ HTa baculovirus expression vector (Gibco/BRL). For HCV genotype 1a, the DNA encoding NS3-NS4A heterodimer protein was PCR-amplified (forward primer: 5′CTCTCTAGATCAGCACTCTTCCATTTCATCGMCTC3′ (SEQ ID No.3); reverse primer: 5′CTCGGATCCGGCGCCCATCACGGCCTACTCCCAA3′ (SEQ ID No.4)) from the HCV genotype la strain H77 (provided by ViroPharma Inc., Exton, Pa., U.S.) and subcloned as described above. The cloning into the pFastBac™ HTa baculovirus expression vector generated a recombinant fusion protein containing an additional N-terminal 28 residues that comprised a hexahistidine tag and a rTEV protease cleavage site. The Bac-to-Bac™ baculovirus expression system (Gibco/BRL) was used to produce the recombinant baculovirus for protein expression.

His-tagged NS3-NS4A heterodimer protease was expressed by infecting Sf21 insect cells (Invitrogen) at a density of 10⁶ cells/mL with the recombinant baculovirus at a multiplicity of infection of 0.1-0.2 at 27° C. The infected culture was harvested 48 to 64 h later by centrifugation at 4° C. The cell pellet was homogenized in 50 mM sodium phosphate, pH 7.5, 40% glycerol (w/v), 2 mM β-mercaptoethanol. His-NS3-NS4A heterodimer protease was then extracted from the cell lysate with 1.5% NP-40, 0.5% Triton X-100, 0.5M NaCl, and a DNase treatment. After ultracentrifugation (100,000×g for 30 min at 4° C.), the soluble extract was diluted 4-fold in 50 mM sodium phosphate, pH 7.5, 0.5M NaCl and loaded on a Pharmacia Hi-Trap Ni⁺²-chelating column. The His-NS3-NS4A heterodimer protein was eluted using a 50 to 400 mM imidazole gradient prepared in 50 mM sodium phosphate, pH 7.5, 10% (w/v) glycerol, 0.1% NP-40, 0.5M NaCl. Co-purification of mature NS3 and NS4A protein complex was verified by Western blot analysis of the purified proteins using an anti-NS3 and an anti-NS4A antiserum produced in-house. The purified NS3 protease domain and the peptide H₂N—PDREVLYREFDEMEEC—OH (SEQ ID No. 5) were used to immunize rabbits for the production of antisera specific to NS3 and NS4A respectively. The proper N-terminal amino acid of both proteins was confirmed by N-terminal amino acid sequencing (PE Biosystems 491/491C Procise Sequencer). The purified enzymes were stored at −80° C. in 50 mM sodium phosphate, pH 7.5, 10% (w/v) glycerol, 0.5 M NaCl, 0.25 M imidazole, 0.1% NP-40.

HCV NS3/4A 2b and 3a

The NS3-NS4 protease genes of HCV genotypes 2b and 3a were amplified from RNA isolated from clinical samples of HCV-infected patients obtained from Dr. G. Steinmann (Germany) using RT-PCR. The primers used for the RT-PCR were 5′CTCGGATCCGGCTCCCATTACTGCTTAC3′ (SEQ ID No. 6) as the forward and 5′GACGCGTCGACGCGGCCGCTCAGCACTCTTCCATTTCACTGM3′ (SEQ ID No.7) as the reverse primer for the NS3-NS4A of HCV genotype 2b and; 5′CTCGGATCGGGCCCCGATCACAGCATACGCC3′ (SEQ ID No. 8) as the forward and 5′CACCGCTCGAGTCAGCATTCTTCCATCTCATCATATTGTTG3′ (SEQ ID No. 9) as the reverse primer for HCV genotype 3a. Each primer contained a unique restriction site for subcloning the fragment in the bacterial expression vector pET11a. The cloning into the vector generated a recombinant fusion protein containing an additional N-terminal 28 residues that comprised a hexahistidine tag and a rTEV protease cleavage site. The recombinant plasmids were used to transform the bacterial strain BL21 DE3 pLysS for protein expression and recombinant protein expression was induced by the addition of IPTG. Following expression, the cells were collected by centrifugation at 4° C. and the cell pellet lysed in a buffer containing 50 mM sodium phosphate, 0.5M NaCl, 40% glycerol, 1.5% NP-40, 0.5% Triton X-100 and the soluble protein fraction was passed through a 5ml HiTrap chelating affinity column as described above. A poly(U)-Sepharose purification step can optionally be conducted in order to remove degradation products and contaminants.

In Vitro HCV NS3 Protease Assay

The fluorogenic depsipeptide substrate used to assess inhibition of protease activity of the HCV 1a, 1b, 2b, 2a-c and 3a NS3-NS4A heterodimer protein was anthranilyl-DDIVPAbu[C(O)—O]-AMY(3-NO₂)TW—OH (SEQ ID No.10). This substrate is cleaved between the aminobutyric (Abu) and the alanine residues. The sequence DDIVPAbu-AMYTW (SEQ ID No.11) is derived from the sequence DDIVPC—SMSYTW (SEQ ID No.12) corresponding to the NS5A/NS5B natural cleavage site. The introduction of the aminobutyric residue at position P1 resulted in a significant decrease of the N-terminal product inhibition while the deletion of the serine residue at position P3 improved the internal quenching efficiency. The protease activity was assayed in 50 mM Tris-HCl, pH 8.0, 0.25M sodium citrate, 0.01% n-dodecyl-β-D-maltoside, 1 mM TCEP. The assay was performed in a Microfluor®2 White U-Bottom Microtiter® plate. 5 μM of the substrate and various concentrations of the test compound were incubated with 1.5 nM of HCV 1a or 1b NS3-NS4A heterodimer protein for 45 minutes at 23° C. under gentle agitation. The final DMSO content did not exceed 5.25%. The reaction was terminated by the addition of a 1M 2-[N-morpholino]ethanesulfonic acid solution at pH 5.8.

The fluorescence was monitored using the BMG POLARstar Galaxy 96-well plate reader with an excitation filter of 320 nm, and an emission filter of 405 nm. The level of inhibition (% inhibition) of each well containing test compound was calculated with the following equation (FU=fluorescence unit):

$$\% \cdot inhibition = ( 1 - \frac{FU \cdot well - FU \cdot blank}{FU \cdot control - FU \cdot blank} ] ) * 100.$$

The calculated % inhibition values were then used to determine IC₅₀, slope factor (n) and maximum inhibition (Iₘₐₓ) by the non-linear regression routine NLIN procedure of SAS using the following equation:

$$\% \cdot inhibition = \frac{{I}_{max} \times {[ inhibitor ]}^{n}}{{[ inhibitor ]}^{n} + {IC}_{{50}^{n}}} .$$

Inhibition Constant (Ki) Determination and mode of Inhibition

The fluorogenic depsipeptide substrate anthranilyl-D(d)EIVP-Nva[C(O)—O] AMY(3-N₂)TW—OH (SEQ ID No.13) was used to assess the mechanism of inhibition and the inhibition constants of the compound (IV) against HCV NS3-NS4A proteases. It was cleaved between the norvaline and the alanine residues. The substrate working solution was prepared in DMSO at the concentration of 200 μM from the substrate stock solution (2 mM in DMSO stored at —20° C.). The final substrate concentration in the assay varied from 0.25 to 8 μM. The inhibition constant (Kᵢ) for compound (IV) was determined using a steady-state velocity method (Morrisson et al. 1985). The protease activity was determined by monitoring the fluorescence change associated with the cleavage of the internally quenched fluorogenic substrate using a SLM-AMINCO® 8100 spectrofluorometer (emission at 325 nm and excitation at 420 nm). The cleavage reaction was monitored in the presence of 0.25 to 8 μM of substrate, 0.3 nM of HCV 1a or 1b NS3-NS4A heterodimer protein and various concentrations of the test compound IV in 50 mM Tris-HCl, pH 8.0, 0.25M sodium citrate, 0.01% n-dodecyl-β-D-maltoside, 1 mM TCEP. The steady-state analysis of inhibition of the HCV genotype 1a and 1b NS3-NS4A heterodimer proteases were performed using the Dixon and Cornish-Bowden graphical methods. In the Dixon graphical method, the reciprocal velocity 1/V is plotted against the test compound concentration at several substrate concentrations (S). In the Cornish-Bowden graphical method, the ratio S/V is plotted against the test compound concentration at several S concentrations. For both methods, the points lie on a straight line at each S value. For a competitive test compound, the Dixon plot displays lines at different S values intersecting at a single point, while the Cornish-Bowden plot displays parallel lines at different S values. The Kᵢ was estimated by fitting the data to the equation describing competitive binding:

$$V = \frac{{k}_{cat} \cdot [ E ] \cdot [ S ]}{{K}_{m} \cdot ( 1 + [ I ] / {K}_{i} ) + [ S ]} .$$

Inhibition of HCV NS3 Proteases

The activity of HCV genotype 1a, 1b, 2b, 2a-c and 3a NS3/NS4A heterodimer proteases was determined in the presence of compound (IV) using the in vitro enzymatic fluorogenic assay. The IC₅₀ curves were analyzed individually by the SAS NLIN procedure. For HCV 1a NS3-NS4A protease, an average IC₅₀ value of 4.3 nM was obtained from the analysis of two batches of compound (IV). An average IC₅₀ value of 3.4 nM was obtained from the analysis of two batches of compound (IV) in the presence of HCV 1b NS3-NS4A protease.

TABLE 1
IC₅₀ of different NS3 protease inhibitors in HCV of different genotypes

| compound | HCV-1a | HCV-1b | HCV-3a | HCV-2a-c | HCV-2b |
|---|---|---|---|---|---|
| II | 7.8 nM | 8.9 nM | 360 nM | 680 nM | 1000 nM |
| III | 4.1 nM | 4.2 nM | 190 nM | 490 nM | 510 nM |
| IV | 4.3 nM | 3.4 nM | 280 nM | 200 nM | 260 nM |
| V | 23 nM | 10 nM | 930 nM | 1800 nM | 3000 nM |

n/d = not done

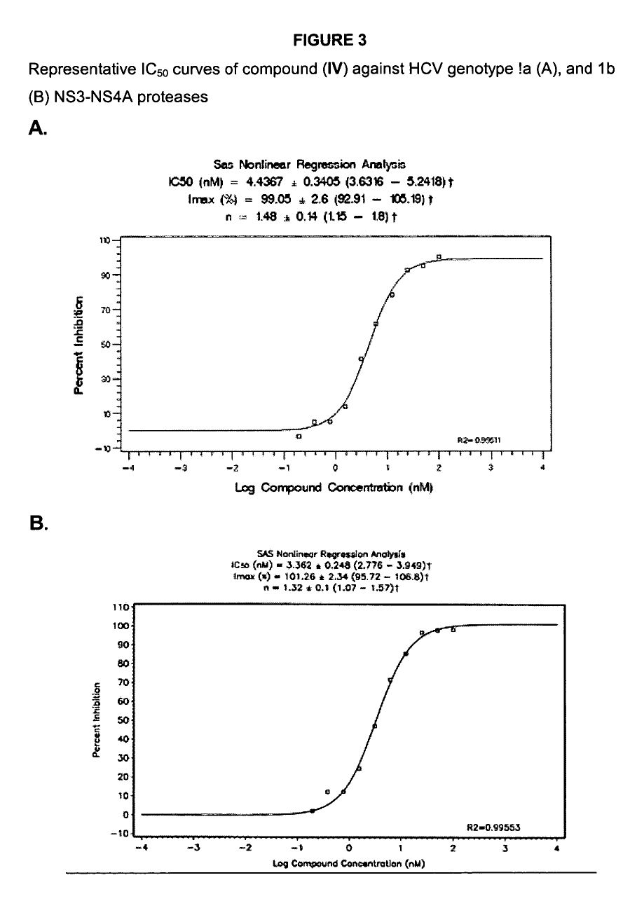

FIGS. 3A and 3B illustrate the IC₅₀ curves of compound (IV) against HCV 1a and 1 b NS3-NS4A proteases, respectively.

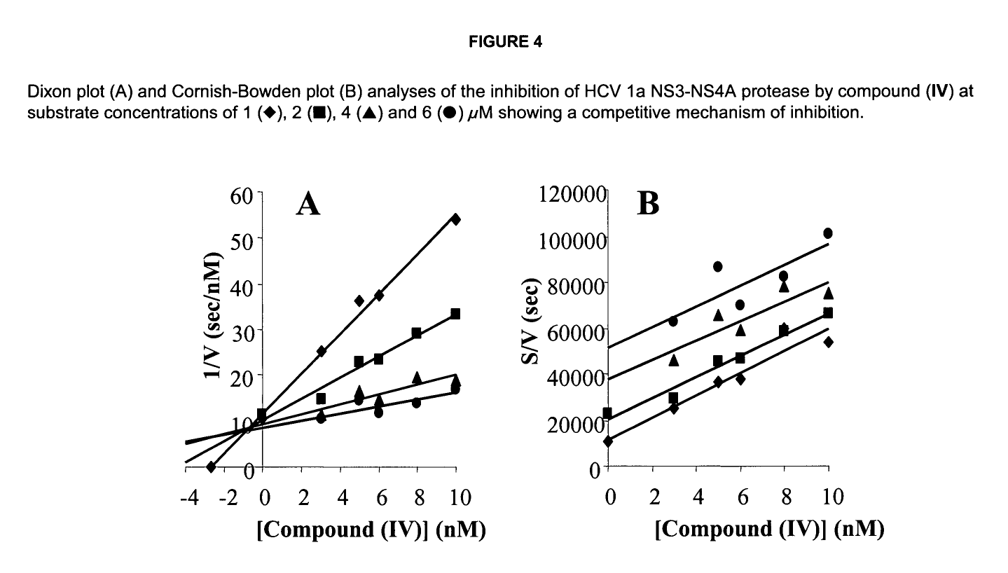

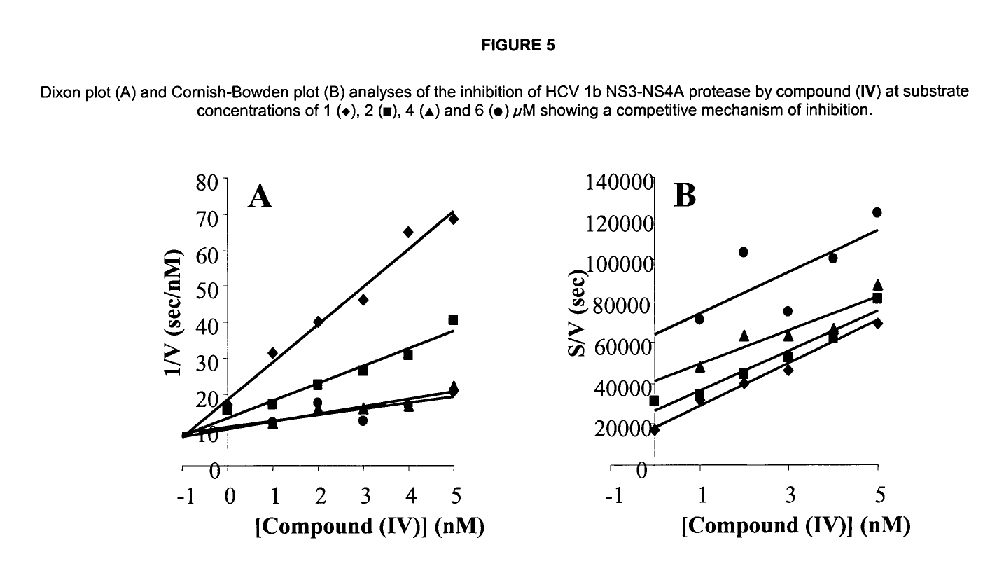

Compound (IV) was found to be a competitive test compound of HCV genotype 1a and 1 b NS3-NS4A proteases following fitting of the data to the equation describing competitive binding with GraFit and also from the Dixon and Cornish-Bowden graphical methods. For both HCV genotype enzymes, the Dixon plot showed that the lines at different S values were intersecting at a single point, while the Cornish-Bowden plot showed parallel lines at various S values. A Kᵢ of 0.30 nM was obtained for compound (IV) from steady-state velocity analysis with the HCV genotype 1a NS3-NS4A protease, while a Kᵢ of 0.66 nM was obtained with the HCV genotype 1b NS3-NS4A protease, Kᵢ's of 90 nM, 86 nM and 83 nM with HCV genotype 3a, 2a-c and 2b respectively. FIGS. 4 and 5 are the Dixon and Cornish-Bowden plots of compound (IV) against the HCV genotype 1a and 1b NS3-NS4A, proteases respectively.

### Example 2

### Inhibition of HCV Replicon 1a and/or 1b

Cell-Based HCV 1a and 1b RNA Replicon Assays

HCV RNA replication was demonstrated in HCV 1a and 1b replicon-containing, Huh-7-derived cell lines developed at Boehringer Ingelheim (Canada) Ltd., R & D (WO 02/052015 incorporated herein by reference). These cells were used to establish a sensitive HCV RNA replication cell-based assay for testing candidate test compounds.

Huh7 cells that stably maintain a subgenomic HCV replicon were established as previously described (Lohman et al. 1999; WO 02/052015). The cells were seeded into a 96 well cell culture cluster at 1×10⁴ cells per well in DMEM complemented with 10% FBS, PenStrep (Life Technologies) and 1 μg/mL Geneticin. Cells were incubated in a 5% CO₂ incubator at 37° C. until addition of various concentrations of the test compound.

The test compound was prepared for use in the assay as follows. The test compound in 100% DMSO was added diluted in assay Medium for a final DMSO concentration of 0.5% and the solution was sonicated for 15 min and filtered through a 0.22 μM Millipore Filter Unit. Serial dilutions of the test compounds were prepared using Assay Medium (containing 0.5% DMSO).

Cell culture medium was aspirated from the 96-well plate containing the cells and the appropriate dilution of test compound in assay medium was transferred to independent wells of the cell culture plate which was incubated at 37° with 5% CO₂.

Following a 3 day-incubation period, the cells were washed with PBS and total cellular RNA was extracted with the RNeasy Mini Kit® and Qiashredder® from Qiagen. RNA from each well was eluted in 50 μL of H₂O. RNA was quantified by optical density at 260 nm on the Cary 1E® UV-Visible spectrophotometer. The HCV RNA replicon copy number was evaluated by real-time RT-PCR with the Abi Prism® 7700 Sequence Detection System. The TAQMAN E® RT-PCR kit provides a system for the detection and analysis of RNA. Direct detection of the reverse transcription polymerase chain reaction (RT-PCR) product with no downstream processing was accomplished by monitoring the increase in fluorescence of a dye-labeled DNA probe. The nucleotide sequence of both primers and probe was located in the 5′ region of the HCV genome. The replicon copy number was then evaluated using a standard curve made with known amounts of replicon copy (supplemented with 50 ng of wild type Huh-7 RNA) assayed in the same reaction mix. The following thermal cycle parameters were used for the RT-PCR reaction on the ABI Prism® 7700 Sequence Detection System. Conditions were optimized for HCV detection. Quantification is based on the threshold cycle, where the amplification plot crosses a defined fluorescence threshold. Comparison of the threshold cycles provides a highly sensitive measure of relative template concentration in different samples. Monitoring during early cycles, when PCR fidelity is at its highest, provides precise data for accurate quantification. The relative template concentration can be converted to real numbers by using the standard curve of HCV with known number of copy.

EC₅₀ Curve Fitting with NLIN Procedure of SAS

The level of inhibition (% inhibition) of each well containing test compound was calculated with the following equation (CN═HCV Replicon copy number):

$$\% \cdot inhibition = ( \frac{CN \cdot control - CN \cdot well}{CN \cdot control} ) * 100.$$

The calculated % inhibition values were then used to determine EC₅₀, slope factor (n) and maximum inhibition (Iₘₐₓ) by the non-linear regression routine NLIN procedure of SAS using the following equation:

$$\% \cdot inhibition = \frac{{I}_{max} \times {[ inhibitor ]}^{n}}{{[ inhibitor ]}^{n} + {EC}_{{50}^{n}}} .$$

TABLE 2
EC₅₀ (nM) of different NS3 protease
inhibitors in HCV of different genotypes

| compound | HCV replicon 1a | HCV replicon 1b |
|---|---|---|
| II | 34 | 27 |
| III | 4.5 | 4.7 |
| IV | 2.1 | 1.0 |
| V | 76 | 39 |
| VI | 2.2 | 3.0 |
| VII | 3.2 | 3.3 |
| VIII | 0.7 | 1.1 |

In HCV replicon cell-based assays, compound (IV) showed a dose-dependent inhibition of HCV RNA replication with EC₅₀s of 2.1 nM and 1 nM using HCV replicon-containing cells of genotype 1a and 1b, respectively. Cytotoxicity was not observed at concentrations higher than 1000 nM. These results indicate that compounds II, III, IV, VI, VII and VIII are potent and specific inhibitors of cell-based HCV RNA replication. Compound V is a compound from a different class but which is related in structure, and also active against NS3 protease but less potent. This compound will be used later on as a control to compare inhibitory activity levels.

Compounds VI, VII and VIII have the following structures:

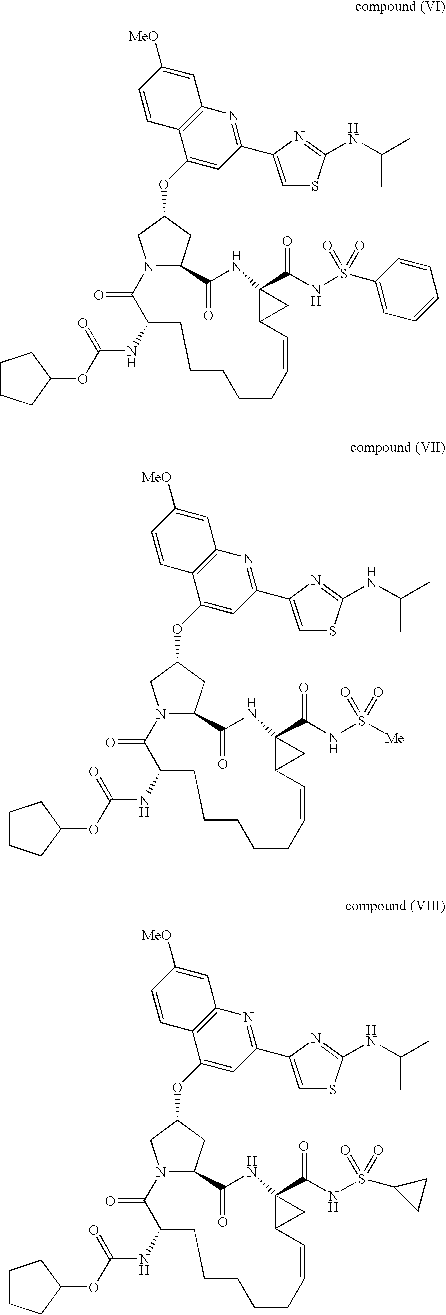

### Example 3

### Inhibition of GBV-B NS3/4A Protease by Compounds II, III and IV

Cloning of GBV-B NS3/NS4A Protease

The full length GBV-B NS3/NS4A protease gene was isolated from infected tamarin serum. Total RNA was isolated from the serum using Qiagene viral RNA extraction kit according to standard protocol. The selection of primers for the reverse transcriptase and the PCR reactions were selected based on the published sequence (Muerhoff, A. S et al. 1995) and GeneBank accession number U22304 (forward: 5′CGCATATGGCACCTTTTACGCTGCAGTGTC3′ (SEQ ID No.14); reverse: 5′CGCGCGCTCGAGACACTCCTCCACGATTTCTTC3′ (SEQ ID No.15)). The amplified RT-PCR product was cloned into the polyhistidine tag-containing pET-29 plasmid between the NdeI and XhoI sites in frame with the polyhistidine tag. This construct produced a recombinant protein with a poly His tag at its C-terminus. The plasmid was transformed into BL21 DE3 pLysS for protein expression.

The E. coli clone pGBV-B was grown in LB medium to a cell density (OD₆₀₀) of 0.6 at which time IPTG was added at a concentration of 0.2 mM. The induction was done at 23 C for a period of 4 hours. The GBV-B NS3/NS4A-His protein was purified according to the procedure described by Zhong et al., 1999. The soluble fraction was purified on a Pharmacia® Hi-Trap Ni⁺²-chelating affinity column using a 50 to 400 mM imidazole gradient. This step was followed by chromatography of the protein preparation on a Superdex® 200 gel filtration column. The concentration of the protein preparation was determined by Bradford protein assay.

In vitro GBV-B NS3 Protease Assay

The depsipeptide substrate used to assess inhibition of the protease activity of GBV-B NS3-NS4A heterodimer protein was Ac-DED(EDANS)EE-Abu[C(O)—O] ASK(DABCYL)-NH₂ (SEQ ID No. 16) This substrate is cleaved between the aminobutyric (Abu) and the alanine residues and products of the reaction were analyzed by HPLC. The protease activity was assayed in 50 mM Tris-HCl;, pH 8.0, 0.25M sodium citrate, 0.01 % n-dodecyl-β-D-maltoside, 1 mM TCEP. The assay was performed in a Microfluor®2 White U-Bottom Microtiter® plate. 5 μM of the substrate and various concentrations of the test compounds were incubated with 0.4 nM of GBV-B NS3-NS4A heterodimer protein for 2 hours at 23° C. under gentle agitation. The final DMSO content did not exceed 5.25%. The reaction was terminated by the addition of a 1M 2-[N-morpholino]ethanesulfonic acid solution at pH 5.8. For quantification, the cleavage products and the substrate were separated by HPLC on a Perkin-Elmer® 3×3CR C8 column. The separation was accomplished by initially eluting at 3.5 mL/min with an aqueous solution containing 0.05% phosphoric acid and 3 mM SDS. A 0 to 45% acetonitrile linear gradient in 0.05% phosphoric acid was then applied for 10 minutes. The absorbance was monitored at 210 nm.

The assay was conducted in the presence of the test compounds II, III, IV and V. The IC₅₀ obtained for each test compound in the presence of GBV-B NS3-4A protein is indicated in Table 3.

TABLE 3

| | IC₅₀ (μM) |
|---|---|
| Compounds | GBV-B |
| compound (II) | 36.5 |
| compound (III) | 31 |
| compound (IV) | 16 |
| compound (V) | 63 |

Compound V is a compound from a different class which is related in structure and also active against NS3 protease but less active than compounds II, III and IV. This compound was included to demonstrate that the ranking of the activity of compounds II, III and IV was maintained between HCV and GBV-B.

Inhibition of GBV-B Replication in Tamarin Hepatocytes in Culture

Hepatocytes isolated from uninfected tamarins were maintained in culture in defined medium for several days. The cell culture model was as described by Beames et al. 2000. Three days after plating, the cells were infected with GBV-B-containing plasma. After viral adsorption, the virus was removed and the cells were incubated in the presence and absence of candidate test compounds at a concentration of 10 μM. The cells were harvested 7 days post infection. The levels of GBV-B RNA were quantitated from total cellular RNA by a real-time PCR assay using a primer-probe combination that recognized a portion of the GBV-B capsid gene (Beames et al. 2000).

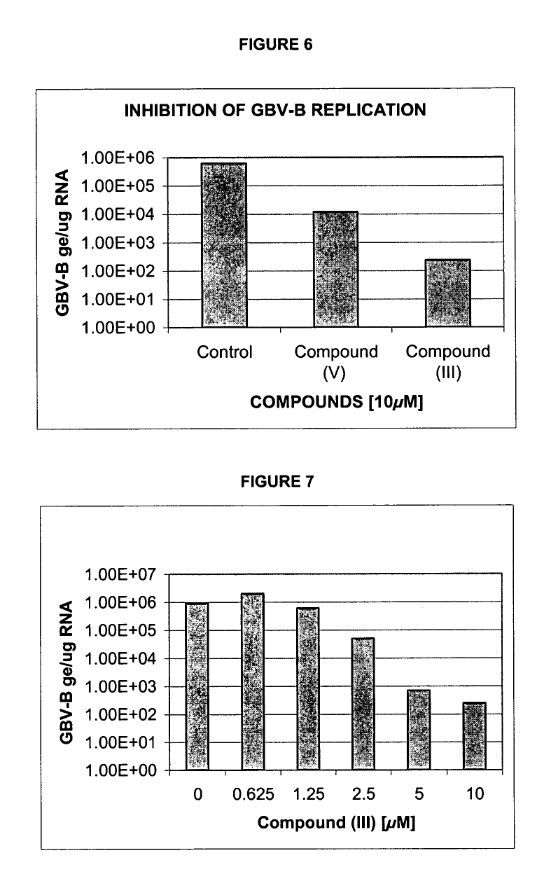

Despite lower IC₅₀ obtained in the enzymatic assay, the results illustrated in FIG. 6, show that more than a 3-log reduction in the viral RNA levels was observed when the cells were incubated in the presence of compound III and a 2-log reduction in the presence of compound V (when the results were expressed as g.e. (genome equivalents)/μg RNA). The results obtained with this more representative viral replication assay demonstrate that these compounds are effective antivirals to reduce GBV-B virus production.

### Example 4

### Sequence Comparison of NS3 Proteases within the Flaviviridae Family

The sequences of proteases from other viruses of the flaviviridae family of viruses were obtained from a BLAST analysis (Altschul et al.1997) NCBI followed by the taxonomy report. The search for protease sequences was done using specific criteria that would retrieve all flaviviridae sequences with similarity to HCV protease excluding HCV sequences themselves. When possible an “NP_” sequence from NCBI was used to represent a particular group of related viruses since these “NP_” sequences are “Reference” sequences (RefSeq). Furthermore, when not known, the NS3 N-terminal and C-terminal ends for the proteases were determined from similarity with HCV NS3 protease.

Sequence alignments of proteases for the six HCV strains against the different groups of viruses were performed using ALIGNX (VectorNTI®) based on CLUSTALW (Thompson, J D, et al., 1994).

From these alignments, the percentage of similarity was calculated between all individual sequences and expressed in Tables 4-8. The similarity was based on the following grouping of amino acids; [ILVM], [FWY], [KR], [DE], [GA], [NQ], [ST].

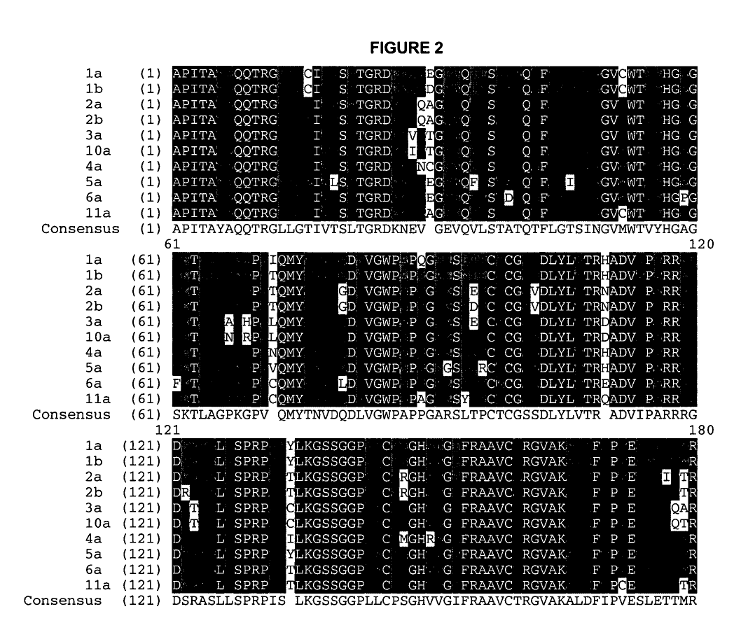

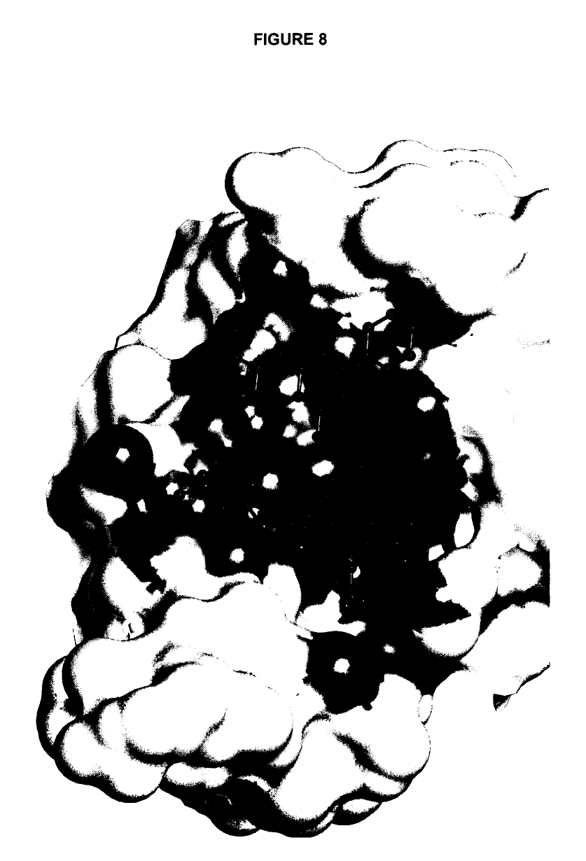

Table 4 represents identities and similarities between several genotypes and subtypes of HCV. One can see that identities range between 70% and 89% whereas similarities range between 77% and 95% demonstrating that the NS3 protease is highly conserved among HCV. Also, from the amino acid sequence alignment of NS3 protease domain of various representative HCV genotypes (FIG. 2) and location of the conserved residues on the three dimensional structure (FIG. 8), one can see that the residues at the active site are highly conserved among HCV genotypes and subtypes.

Table 5 is an analysis of the similarity between GB viruses NS3 protease and HCV NS3 protease domains. Similarities range between 43 and 49%, whereas similarities between HCV and pestivirus NS3 domains range from 25 and 30% (Table 6). Similarities between HCV and dengue virus range also around 30% (Table 7), as well as the similarity between HCV and Flavivirus (Table 8).

In contrast, Table 9 shows that the similarities between the NS3 protease domain of HCV and the protease of Human Cytomegalovirus (HCMV) revolves around 20%. HCMV is a virus of the Herpes viridae and has a serine protease that has a different catalytic triad from Flaviviridae proteases. The HCMV virus protease is therefore not considered to be similar to Flaviviridae NS3 proteases. This contrast will be illustrated further when comparison is made of the similarities between the amino acid in contact with our inhibitors in Flaviviridae and HCMV.

### Example 5

### Three-Dimensional Structural Crystal Studies

In order to analyze further the similarities between Flaviviridae, a crystal structure was obtained of the HCV NS3 protease complexed with an inhibitor, and the amino acids directly in contact with the inhibitor were analyzed in other Flaviviridae to assess degrees of similarity of these important contact points.

A co-crystal of the HCV NS3 protease-NS4A peptide complexed with macrocyclic compound (II) was obtained that diffracted X-rays to 2.75 Å resolution. The macrocyclic compound II was found at the NS3 protease active site and was clearly defined in the difference electron density. This structure reveals molecular details of how the test compound interacts with the active site and provides additional insight into the mechanism of inhibition of the HCV NS3 protease.

The structure of the NS3 protease complex with the test compound II (FIG. 8) better defines the binding site of the present substrate-based test compound series. From this structure of the co-complex, the residues directly in contact with the test compound i.e. within 3 Å (H57, G137, S139, A156 and A157) are indicative of the active site, as predicted by the competitive mode of inhibition, and by the conservation at this site among representative sequences of HCV genotypes and various subtypes.

Other residues in contact within 4 Å of the test compound are listed in Table 10. Comparison analysis reveals that these amino acids are highly conserved among Flaviviridae ranging between 59% and 76%. Conservation of these residues among Flaviviridae is indicative that inhibitors of the HCV NS3 protease would also inhibit other members of the Flaviviridae family of viruses. In contrast, the level of similarity of the HCMV protease catalytic site and the contact points of Table 10 is in the same range as the similarity for the whole protease (˜20%) once again illustrating the fact that other serine protease from non-Flaviviridae are not targeted by the compounds of formula (I).

Other structural studies performed between HCV and dengue virus protease domains have shown that all six strands of the C-terminal domain inhibitor binding site are strongly conserved and of comparable length. Superposition of 68 α carbon atoms of the C-terminal domain of Den2 protease (residues 87-167) and HCV including most of the residues in Table 10 (residues 69-189) yields a r.m.s. deviation of 0.9 Å. (Murthy et al., 1999).

Discussion

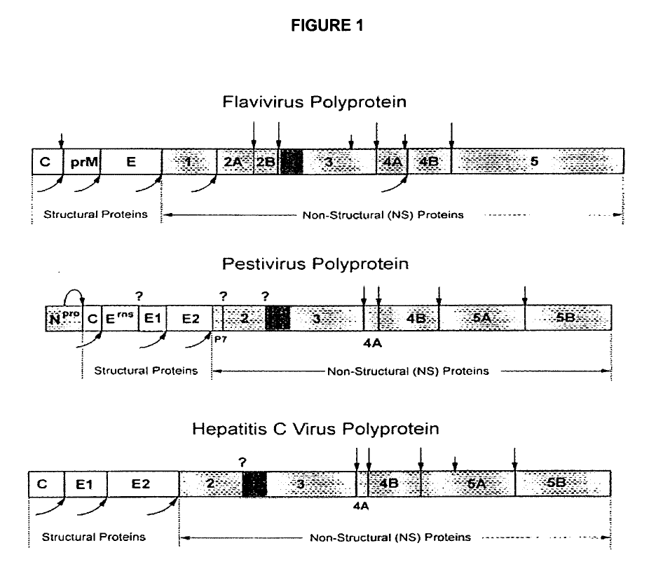

Viruses within the Flaviviridae family possess a number of similarities (FIG. 1) despite a relatively low overall sequence homology among its members (Tables 4-8). In particular, the NS3 protein contains sequences with similarity to the protease and the nucleoside triphosphate-binding helicase of pestiviruses and flaviviruses. The HCV NS3 protease domain shares a sequence similarity of about 77-95% among HCV genotypes (FIG. 1) and a sequence similarity of about 25-50% with other members of the Flaviviridae family (Tables 4-8). In addition, the genomic organization and structure of GBV-B and HCV are similar despite the fact that the sequence homology between the polyprotein sequences of GBV-B and HCV is about 25 to 30%. This low similarity is however trumped by the fact that residues that come in contact with the inhibitor are highly conserved within the protease domain of Flaviviridae, a strong indication that this family of compounds would also bind to other Flaviviridae. The activity of compound IV obtained against GBV-B is a positive demonstration of that hypothesis.

With respect to the three dimensional structure, the available atomic co-ordinates of the various crystallized HCV and Dengue NS3 proteases showed an overall architecture that is characteristic of the trypsin-like fold. Many studies have now firmly established that the N-terminal portion of the NS3 region encodes a serine protease that has a very specific and pivotal role in viral polyprotein processing within Flaviviridae.

All results produced in the above experiments tend to support the contention that the compounds of formula (I) are active against members of the Flaviviridae family of viruses:
6 related compounds have been shown to be active against HCV 1b and have similar activity against HCV 1a (Table 2);
3 of these compounds were also tested against HCV 3a, 2a-c and 2b and also found to be active (Table 1);
these 3 same compounds were tested in the enzymatic assay against GBV-B and found to be active (Table 3);
1 of these compounds was tested in cell culture against replication of GBV-B in tamarin cells and found to be active (FIG. 6);
1 of these compounds demonstrates a mode of binding that is situated in a region highly conserved between all members of Flaviviridae (FIG. 8);
studies have demonstrated strong functional and structural similarity between HCV and Dengue virus NS3 proteases.

Given all similarities of viruses within the Flaviviridae family of viruses, it seems highly likely that the compounds of Formula (I) are effective against members of the Flaviviridae family of viruses, and more particularly, effective against the pathogenic members of the Flaviviridae family.

References

Altschul S. F. et al. 1997, Nucleic Acids Res. 25, 3389-3402
Ausubel et al. 1994, Current Protocols in Molecular Biology, Wiley, New York
Beames, B. et al. 2000, J. Virol., 74,11764-11772
Butriewicz, N. et al. 2000, J. Virol., 74,4291-4301
Kim J L, et al. 1996, Cell;87:343-355.
Lin, C. et al. 1995, J. Virol. 69,4373-4380
Lohmann et al. 1999, Science 285, 110
Love R. A. et al. 1996 Cell 87, 331-342
Morrison, J. F. and Stone, S. R. 1985, Mol. Cell. Biophys. 2, 347-368
Muerhoff, A. S. et al. 1995 J. Virol.69, 5621-5630
Murthy, H. M. et al. 1999, J. Biol. Chem. 274(9), 5573-5580
Remington, 1995, The Science and Practice of Pharmacy
Ryan, M. D. et al. 1998 J. Gen. Virol. 79, 947-959
Sambrook et al. 1989, Molecular Cloning—A Laboratory Manual, Cold Spring Harbor Labs
Scarselli, E., et al. 1997, J. Virol. 71, 4985-4989
Steinkuler, C. et al. 1996, J. Biol. Chem. 271, 6367-6373.
Thompson, et al. 1994, Nucleic Acids Res. 22: 4673-4680

Zhong, W. et al. 1999, Virology 261, 216-226

TABLE 4
IDENTITIES AND SIMILARITIES BETWEEN NS3 PROTEASE

| | 1_1a | 1_1b | 2_2a | 2_2b | 3_3a | 3_10a | 4_4a | 5_5a | 6_6a | 6_11a |
|---|---|---|---|---|---|---|---|---|---|---|
| 1_1a | | I = 88% | I = 70% | I = 71% | I = 76% | I = 72% | I = 81% | I = 80% | I = 81% | I = 79% |
| | | S = 95% | S = 81% | S = 80% | S = 84% | S = 82% | S = 86% | S = 90% | S = 88% | S = 88% |
| 1_1b | | | I = 71% | I = 72% | I = 79% | I = 76% | I = 83% | I = 80% | I = 82% | I = 81% |
| | | | S = 82% | S = 82% | S = 87% | S = 85% | S = 88% | S = 91% | S = 90% | S = 90% |
| 2_2a | | | | I = 87% | I = 74% | I = 71% | I = 72% | I = 71% | I = 74% | I = 74% |
| | | | | S = 95% | S = 81% | S = 81% | S = 82% | S= 82% | S = 86% | S = 86% |
| 2_2b | | | | | I = 70% | I = 69% | I = 72% | I = 71% | I = 73% | I = 71% |
| | | | | | S = 78% | S = 77% | S = 81% | S = 81% | S = 83% | S = 83% |
| 3_3a | | | | | | I = 89% | I = 75% | I = 76% | I = 79% | I = 75% |
| | | | | | | S = 93% | S = 82% | S = 83% | S = 87% | S = 83% |
| 3_10a | | | | | | | I = 75% | I = 76% | I = 77% | I = 75% |
| | | | | | | | S = 82% | S = 83% | S = 85% | S = 82% |
| 4_4a | | | | | | | | I = 79% | I = 79% | I = 76% |
| | | | | | | | | S = 85% | S = 86% | S = 85% |
| 5_5a | | | | | | | | | I = 82% | I = 78% |
| | | | | | | | | | S = 89% | S = 87% |
| 6_6a | | | | | | | | | | I = 80% |
| | | | | | | | | | | S = 88% |

Similarity score was obtained defining the following amino acids similarity: [ILVM], [FWY], [KR], [DE], [GA], [NQ], [ST] domains (180aa) of the different HCV genotypes and subtypes

TABLE 5
% SIMILARITY OF HCV NS3 PROTEASE DOMAIN (181 AA) WITH
GB NS3 PROTEASES FROM THE FLAVIVIRIDAE FAMILY

| | GBV-B | HepG | GBV-A | GBV-C |
|---|---|---|---|---|
| | NP_056931 | NP_043570 | NP_045010 | NP_059446 |
| 1a | 45 | 49 | 44 | 48 |
| AF271632 | | | | |
| 1b | 46 | 49 | 45 | 48 |
| D90208 | | | | |
| 2a | 44 | 48 | 45 | 47 |
| D00944 | | | | |
| 2b | 46 | 47 | 45 | 46 |
| D10988 | | | | |
| 3a | 44 | 45 | 43 | 45 |
| D17763 | | | | |
| 10a | 43 | 45 | 45 | 46 |
| D63821 | | | | |
| 4a | 44 | 46 | 45 | 45 |
| Y11604 | | | | |
| 5a | 46 | 48 | 44 | 46 |
| Y13184 | | | | |
| 6a | 45 | 47 | 46 | 46 |
| Y12083 | | | | |
| 11a | 44 | 49 | 45 | 48 |
| D63822 | | | | |

Matrix: [ILVM], [FWY], [KR], [DE], [GA], [NQ], [ST]

TABLE 6
% SIMILARITY OF HCV NS3 PROTEASE DOMAIN
(181 AA) WITH PESTIVIRUSES NS3 PROTEASES
FROM THE FLAVIVIRIDAE FAMILY

| | Pesti 1 | BVDV2 | Pesti 2 | Pesti 3 |
|---|---|---|---|---|
| | NP_040937 | NP_044731 | NP_075354 | NP_620062 |
| 1a | 28 | 28 | 28 | 28 |
| AF271632 | | | | |
| 1b | 28 | 28 | 28 | 28 |
| D90208 | | | | |
| 2a | 26 | 26 | 26 | 26 |
| D00944 | | | | |
| 2b | 26 | 25 | 26 | 26 |
| D10988 | | | | |
| 3a | 29 | 28 | 28 | 28 |
| D17763 | | | | |
| 10a | 28 | 27 | 28 | 28 |
| D63821 | | | | |
| 4a | 28 | 28 | 28 | 28 |
| Y11604 | | | | |
| 5a | 29 | 29 | 29 | 29 |
| Y13184 | | | | |
| 6a | 30 | 29 | 29 | 29 |
| Y12083 | | | | |
| 11a | 29 | 28 | 29 | 29 |
| D63822 | | | | |

BVDV-2 = Bovine viral Diarrhea virus

TABLE 7
% SIMILARITY OF HCV NS3 PROTEASE DOMAIN
(181 AA) WITH MOSQUITO-BORNE FLAVIVIRUS NS3
PROTEASES FROM THE FLAVIVIRIDAE FAMILY

| | Dengue 1 | Dengue 2 | Dengue 3 | Dengue 4 |
|---|---|---|---|---|
| | NP_059433 | NP_056776 | NP_040961 | NP_073286 |
| 1a | 26 | 27 | 28 | 29 |
| AF271632 | | | | |
| 1b | 29 | 29 | 30 | 31 |
| D90208 | | | | |
| 2a | 27 | 29 | 28 | 28 |
| D00944 | | | | |
| 2b | 29 | 29 | 29 | 29 |
| D10988 | | | | |
| 3a | 27 | 29 | 29 | 29 |
| D17763 | | | | |
| 10a | 28 | 29 | 30 | 29 |
| D63821 | | | | |
| 4a | 27 | 27 | 28 | 30 |
| Y11604 | | | | |
| 5a | 29 | 30 | 31 | 30 |
| Y13184 | | | | |
| 6a | 29 | 30 | 30 | 31 |
| Y12083 | | | | |
| 11a | 29 | 29 | 30 | 30 |
| D63822 | | | | |

TABLE 8
% SIMILARITY OF HCV NS3 PROTEASE DOMAIN
(181 AA) WITH FLAVIVIRUS NS3 PROTEASES FROM
THE FLAVIVIRIDAE FAMILY

| | WNV | JEV | Kunjin | YFV | MVEV |
|---|---|---|---|---|---|
| | NP_{—} | NP_{—} | POLG_{—} | NP_{—} | NP_{—} |
| | 041724 | 059434 | KUNJM | 041726 | 051124 |
| 1a | 29 | 30 | 29 | 29 | 30 |
| AF271632 | | | | | |
| 1b | 29 | 31 | 30 | 29 | 31 |
| D90208 | | | | | |
| 2a | 28 | 28 | 28 | 28 | 28 |
| D00944 | | | | | |
| 2b | 29 | 30 | 29 | 30 | 30 |
| D10988 | | | | | |
| 3a | 27 | 27 | 27 | 28 | 28 |
| D17763 | | | | | |
| 10a | 27 | 27 | 27 | 28 | 27 |
| D63821 | | | | | |
| 4a | 28 | 29 | 29 | 29 | 30 |
| Y11604 | | | | | |
| 5a | 29 | 30 | 30 | 30 | 30 |
| Y13184 | | | | | |
| 6a | 29 | 30 | 30 | 28 | 30 |
| Y12083 | | | | | |
| 11a | 28 | 29 | 28 | 29 | 29 |
| D63822 | | | | | |

WVN = West Nile virus
JEV = Japanese encephalitis virus
Kunjin = Kunjin virus
YFV = Yellow Fever virus
MVEV = Murray Valley encephalitis virus

TABLE 9
% SIMILARITY OF HCV PROTEASE DOMAIN (181 AA) WITH
HUMAN CYTOMEGALOVIRUS PROTEASE DOMAIN (256 AA)

| | HCMV protease |
|---|---|
| 1a | 20 |
| AF271632 | |
| 1b | 20 |
| D90208 | |
| 2a | 21 |
| D00944 | |
| 2b | 20 |
| D10988 | |
| 3a | 18 |
| D17763 | |
| 10a | 18 |
| D63821 | |
| 4a | 19 |
| Y11604 | |
| 5a | 19 |
| Y13184 | |
| 6a | 19 |
| Y12083 | |
| 11a | 19 |
| D63822 | |

TABLE 10
AMINO ACID DIFFERENCES IN THE NS3 PROTEASE INHIBITOR
BINDING DOMAIN AMONG VIRUSES OF THE (WITHIN
4 Å OF THE INHIBITOR) FLAVIVIRIDAE FAMILY

| HCV40_{—} | | | | | | 132 | | | |
|---|---|---|---|---|---|---|---|---|---|
| 1b | 56 Y | 57 H | 79 D | 81 D | 123 R | V, I | 135 L | 136 K | 137 G |
| HCV | | | E₂₀ | | T₆ | I₄₉ | | | |
| All | | | | | K₁ | L₂₆ | | | |
| genotypes | | | | | | | | | |
| (140) | | | | | | | | | |
| GBVₐ | F/Y+ | H+ | A/S | D+ | K/Y+ | V/L/M+ | F/A | R/K+ | G+ |
| PESTI | D | H+ | M | D+ | T | L+ | L+ | K+ | G+ |
| Flavivirus/ | W+ | H+ | K | D+ | P/N/L | L+ | K/S | P | G+ |
| Mosquito- | | | | | | | | | |
| borne | | | | | | | | | |
| (Dengue) | | | | | | | | | |
| Flavivirus | W+ | H+ | K | D+ | P/N/ | L+ | P | X | G+ |
| mosquito- | | | | | | | | | |
| borne | | | | | | | | | |
| Herpesvirus | V | R | V | E+ | T | G | A | V | D |
| HCMV | | | | | | | | | |
| HCV40_{—} | | | | | | | | | % |
| 1b | 138 S | 139 S | 154 F | 155 R | 156 A | 157 A | 158 V | 168 D | Sim |
| HCV | | | | | | | | Q₆ | 88-100 |
| All | | | | | | | | E₄ | |
| genotypes | | | | | | | | | |
| (140) | | | | | | | | | |
| GBVₐ | S+ | S+ | L/F= | V/T | S/A= | V/A= | L/R+ | R | 59 |
| | | | | | | | | | 76_{b} |
| PESTI | W | S+ | V | K+ | V | G+ | G | K | 53 |
| Flavivirus/ | T+ | S+ | Y+ | G | N | G+ | V+ | A | 59 |
| Mosquito- | | | | | | | | | |
| borne | | | | | | | | | |
| (Dengue) | | | | | | | | | |
| Flavivirus | T+ | S+ | Y+ | G | N | G+ | V+ | A | 59 |
| mosquito- | | | | | | | | | |
| borne | | | | | | | | | |
| Herpesvirus | A | S+ | S | V | D | A+ | L+ | R | 23 |
| HCMV | | | | | | | | | |

HCV40_1b: Sequence of a HCV 1b isolate that was used as reference for this analysis
n: number of HCV sequences of all genotypes that was used in the study
Aₙ: number of HCV sequences containing this modification
% Sim: % similarity
+Similarity according to Matrix [ILVM], [FWY], [KR], [DE], [GA], [NQ], [ST]
=Similarities that are unique for the GBV-B member of the GBV group
GBVₐ: The aa in bold are the residue found for GBV-B NS3 protease
_{b}% similarity for GBV-B

1. A method for the treatment of a mammal infected with a virus of the Flaviviridae family comprising administering a therapeutically effective amount of a compound of Formula (I): 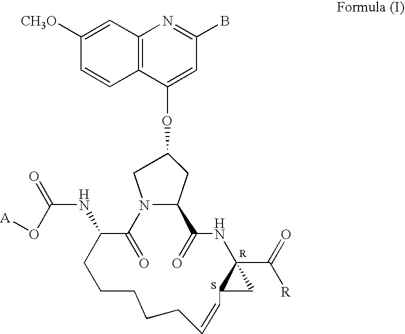
wherein A is selected from: C₁ to C₆ alkyl and C₃ to C₆ cycloalkyl; B is selected from: phenyl or thiazolyl, both of which optionally substituted with a group selected from NH(R¹) and NH(CO)R¹, wherein R¹ is C₁ to C₆ alkyl; and R is OH or a sulfonamide group of the formula —NHSO₂—R² wherein R² is —(C₁₋₈)alkyl, —(C₃₋₇)cycloalkyl or {—(C₁₋₆)alkyl-(C₃₋₆)cycloalkyl}, which are all optionally substituted from 1 to 3 times with halo, cyano, nitro, O—(C₁₋₆)alkyl, amido, amino or phenyl, or R² is C₆ or C₁₀ aryl which is optionally substituted from 1 to 3 times with halo, cyano, nitro, (C₁₋₆)alkyl, O—(C₁₋₆)alkyl, amido, amino or phenyl;
or a pharmaceutically acceptable salt thereof.

2. The method according to claim 1, wherein A of Formula (I) is a branched C₄ to C₆ alkyl or C₄ to C₆ cycloalkyl group, B of Formula (I) is phenyl or a thiazole substituted at position 2 with NH(R¹) or NH(CO) R¹ in which R¹ is a C₁ to C₄ alkyl, and R is OH or a sulfonamide group of formula —NHSO₂—R² wherein R² is —(C₁₋₆)alkyl, —(C₃₋₆)cycloalkyl, both optionally substituted 1 or 2 times with halo or phenyl, or R² is C₆ aryl optionally substituted from 1 or 2 times with halo or (C₁₋₆)alkyl.

3. The method according to claim 2, wherein A is cyclopentyl or tert-butyl, B is a thiazole substituted at position 2 with NH(R¹) or NH(CO) R¹ in which R¹ is a C₁ to C₄ alkyl, and R is OH or a sulfonamide group wherein R² is methyl, cyclopropyl or phenyl.

4. The method according to claim 1, wherein said mammal is a human, said Flaviviridae is Yellow Fever virus and said compound is a compound of formula (I) wherein A is cyclopentyl, B is a thiazole substituted at its 2 position with NHCH(CH₃)₂, and R is OH or a sulfonamide group wherein R² is methyl, cyclopropyl or phenyl.

5. The method according to claim 1, wherein said mammal is a human, said Flaviviridae is West Nile virus and said compound is a compound of formula (I) wherein A is cyclopentyl, B is a thiazole substituted at its 2 position with NHCH(CH₃)₂, and R is OH or a sulfonamide group wherein R² is methyl, cyclopropyl or phenyl.

6. The method according to claim 1, wherein said mammal is a human, said Flaviviridae is Dengue fever virus and said compound is a compound of formula (I) wherein A is cyclopentyl, B is a thiazole substituted at its 2 position with NHCH(CH₃)₂, and R is OH or a sulfonamide group wherein R² is methyl, cyclopropyl or phenyl.

7. The method according to claim 1, wherein said mammal is a human, said Flaviviridae is Japanese Encephalitis virus and said compound is a compound of formula (I) wherein A is cyclopentyl, B is a thiazole substituted at its 2 position with NHCH(CH₃)₂, and R is OH or a sulfonamide group wherein R² is methyl, cyclopropyl or phenyl.

8. The method according to claim 1, wherein said mammal is a human, said Flaviviridae is GB virus A or C, and said compound is a compound of formula (I) wherein A is cyclopentyl, B is a thiazole substituted at its 2 position with NHCH(CH₃)₂, and R is OH or a sulfonamide group wherein R² is methyl, cyclopropyl or phenyl.

9. The method according to claim 1, wherein said mammal is a human, said Flaviviridae is Hepatitis G virus and said compound is a compound of formula (I) wherein A is cyclopentyl, B is a thiazole substituted at its 2 position with NHCH(CH₃)₂, and R is OH or a sulfonamide group wherein R² is methyl, cyclopropyl or phenyl.

10. The method according to claim 1, wherein said mammal is a cattle, said Flaviviridae is BVDV and said compound is a compound of formula (I) wherein A is cyclopentyl, B is a thiazole substituted at its 2 position with NHCH(CH₃)₂, and R is OH or a sulfonamide group wherein R² is methyl, cyclopropyl or phenyl.

11. The method according to claim 1, wherein said mammal is a sheep, said Flaviviridae is border disease virus and said compound is a compound of formula (I) wherein A is cyclopentyl, B is a thiazole substituted at its 2 position with NHCH(CH₃)₂, and R is OH or a sulfonamide group wherein R² is methyl, cyclopropyl or phenyl.

12. The method according to claim 1, wherein said mammal is a pig, said Flaviviridae is Classical Swine Fever Virus and said compound is a compound of formula (I) wherein A is cyclopentyl, B is a thiazole substituted at its 2 position with NHCH(CH₃)₂, and R is OH or a sulfonamide group wherein R² is methyl, cyclopropyl or phenyl.

13. The method according to claim 1, wherein said Flaviviridae virus comprises an NS3 protease comprising amino acid residues selected from: H57, G137, S139, A156 and A157.

14. An article of manufacture comprising packaging material contained within which is a composition effective to inhibit a virus of the Flaviviridae family and the packaging material comprises a label which indicates that the composition can be used to treat infection by a virus of the Flaviviridae family and, wherein said composition comprises a compound of Formula (I) as defined in claim 1.
